(19) United States
(12) Patent Application Publication
(10) Pub. No.: US 2026/0091207 A1
(43) Pub. Date: Apr. 2, 2026

(54) CONTROLLABLY DISINTEGRABLE SELF-EXPANDABLE INGESTIBLE DEVICES

(71) Applicant: EPITOMEE MEDICAL LTD., Caesarea (IL)

(72) Inventors: Valery ARTAMONOV, Ashdod (IL); Gil COHEN, Jerusalem (IL)

(21) Appl. No.: 19/113,326

(22) PCT Filed: Aug. 17, 2023
(86) PCT No.: PCT/IL2023/050872

§ 371 Date: Mar. 19, 2025

Related U.S. Application Data
(60) Provisional application No. 63/376,513, filed on Sep. 21, 2022.
(60) Provisional application No. 63/383,733, filed on Nov. 15, 2022.

(51) Int. Cl. A61M 31/00

(52) U.S. Cl. CPC A61M 31/00; A61M 2205/0216

(57) ABSTRACT

This disclosure concerns expandable devices, specifically self-deployed devices that are designed to be delivered to and deployed in the gastrointestinal tract and configured for controlled disintegration. The devices contain expandable compartments separated by sealing zones that are designed for controlled disintegration upon exposure to liquid, thereby permitting controlled disintegration of the device after a pre-defined time from deployment.

### TECHNOLOGICAL FIELD

This disclosure concerns expandable devices, specifically self-deployed devices that are designed to be delivered to and deployed in the gastrointestinal tract and configured for controlled disintegration.

### BACKGROUND ART

References considered to be relevant as background to the presently disclosed subject matter are listed below:
PCT patent publication WO2016015648
PCT patent publication WO2008062440
PCT patent publication WO2009125432
PCT patent publication WO2013188819
PCT patent publication WO2015026552
PCT patent publication WO2015120471

Acknowledgement of the above references herein is not to be inferred as meaning that these are in any way relevant to the patentability of the presently disclosed subject matter.

### BACKGROUND

Effective and targeted delivery of various compounds and active agents to or across a tissue, for example the lining of the intestinal tract, has proven over the years to be a challenge. Due to mucus secretions that line various tissues for providing a hydrated environment and lubrication of biological surfaces, targeted delivery of active agents often requires the use of mucoadhesive components or other tissue attachment arrangements, which are capable of attaching to the tissue for a pre-defined period of time, thereby providing longer contact time between the tissue and the active agent to allow longer time of absorption of the active agent through and/or to the tissue.

In some approaches, use of deployable devices is made, in which a device is delivered to the vicinity of the target site in a non-deployed form, and deployed to deliver the mucoadhesive-based components to the tissue during such deployment. In the case of delivery to the GI tract, careful consideration needs to be taken to control both deployment at the proper target site and the evacuation of the device after its deployment in order to avoid obstruction of the GI tract.

### GENERAL DESCRIPTION

The present disclosure provides self-expandable devices, which can be administered to a subject and undergo a controlled deployment in the gastrointestinal tract. The devices can be configured to deliver one or more active agents or one or more tissue-attachable layers (or patches), e.g. mucoadhesive materials, to a tissue at the target site. The devices of this disclosure are designed to permit controlled disintegration of the device, or parts thereof, to permit proper and controlled evacuation of the device from the target site once the device has been deployed, for example once a tissue-attachable layer has been delivered and applied onto the tissue at the target site.

The devices of this disclosure are based on the realization that careful construction of the device to permit controlled absorption of liquids, e.g. water, during and after the device's deployment, can be utilized to control the location and rate of disintegration of at least portions of the device, to permit its breakdown into fragments that are more easily evacuated from the target site.

Thus, by one of its aspects, the present disclosure provides an ingestible self-expandable device that has a collapsed state and an expanded state. The device comprises two or more self-expandable compartments, connected to one another by connecting zones. Each compartment is formed out of two, substantially water-insoluble, deformable film portions that are bonded to each other at peripheral segments of the compartments by at least one water-disintegrable sealing composition to define sealing zones. Each sealing zone is a layered structure comprised of the water-insoluble deformable films sandwiching between them at least one water-disintegrable sealing composition layer that bonds the water-insoluble deformable films to one another at the peripheral segments. The two film portions define between them, at areas enclosed by the peripheral segments, an enclosed space of the compartment. Each compartment has one or more liquid-permeable sections and a gel forming material within said enclosed space, the gel forming material is configured for swelling upon contact with liquid, thereby expanding the compartment to irreversibly switch the device from the collapsed state to the expanded state. In devices of this disclosure, the water-disintegrable sealing composition provides mechanical stability to the sealing zones during transition of the device from the collapsed state to the expanded state, and has water-solubility configured to provide controlled disintegration of the sealing zones after the device is expanded into the expanded state, to thereby cause loss of integrity (breakdown and/or disintegration) of the compartments and/or the device after its deployment.

In other words, in the devices of this disclosure, the sealing zones have several functions. First, the sealing zones form the perimeters of the compartments, as the water-insoluble deformable film portions are attached to one another at the peripheral segments of the compartment by the water-disintegrable sealing composition. Further, the sealing zones provide mechanical stability and reinforcement to the compartments during the deployment of the device from the collapsed to the expanded state, thereby enabling to maintain the compartment's integrity during deployment. However, once deployed, the water-disintegrable sealing composition in the sealing zones functions as weaker structural area that permit controlled disintegration of the device after its expansion to enable breakdown of the device into smaller fragments, which can be evacuated from the target site more easily.

The devices of this disclosure are designed to controllably increase in volume, for example to drive a tissue-attachable layer or patch, e.g. a layer of mucoadhesive material, towards the tissue and temporarily apply force thereonto in order to attach or adhere the layer or patch to the tissue at a desired location. Hence, once the device is administered, exposure to the proper conditions for deployment will cause the device to expand and deliver the tissue-attachable layer to the tissue. After delivery of the tissue-attachable layer to the target site, evacuation of the device is typically required; as the device is voluminous, evacuation is facilitated by disintegration.

As noted, the compartments are connected one to the other by connecting zones. The connecting zones are elements of the device, typically integral with the compartments, that form a physical link between adjacent compartments to obtain the overall shape of the device. According to some embodiments, the connecting zones comprise said sealing zones (i.e. the sealing zones being part of the connecting zones).

According to other embodiments, the compartments are connected one to the other by bonding one or more of their respective sealing zones by a water-disintegrable sealing composition. As typically the water-disintegrable sealing composition is present in sealing zones and in the connecting zones, controlled breakdown of both the device and the compartments can be obtained.

According to some embodiments, the compartments are connected one to the other by stacking sealing zones of adjacent compartments one over the other, such that the connecting zones consist of the stacked sealing zones, to form a layered structure of alternating layers of the water-insoluble deformable films and the water-disintegrable sealing compositions.

By some embodiments, the sealing zones comprise a first water-disintegrable sealing composition, and the adjacent compartments are connected one to the other by bonding one or more of their respective sealing zones by a second water-disintegrable sealing composition, the first and second water-disintegrable sealing compositions being different one from the other or being the same.

By controlling various parameters of the water-disintegrable sealing composition, a balance can be obtained between the stability of the water-disintegrable sealing composition during deployment of the device as to maintain the mechanical integrity of the device during this transition, and the disintegration of the sealing zones after the device's deployment.

The term sealing zone is meant to denote a circumferential contour, in which peripheral segments of the water-insoluble deformable film portions are attached to one another by the water-disintegrable sealing composition. As a result of such an attachment, a compartment is formed, in which the segments of the water-insoluble deformable film portions enclosed by the circumferential contour define between them an enclosed space.

As noted, the sealing zones have a layered structure, in which the water-insoluble deformable films define two external layers of the sealing zone, and a layer of the water-disintegrable sealing composition is sandwiched between the films at the peripheral segments of the compartment. As the external layers of the sealing zones are substantially water-insoluble, penetration of water into the sealing zones is permitted only via the small surface perpendicular to the plane of the layers in the sealing zones in which the water-disintegrable sealing composition is exposed to liquid. Hence, control over the exposure of the water-disintegrable sealing composition to water, and therefore the disintegration rate of the sealing zone (and/or the onset of disintegration), can be obtained, inter alia, by control over the surface area of the water-disintegrable sealing composition that is exposed to water. Further control of the disintegration of the sealing zones can be obtained, for example, by controlling the disintegration rate of the water-disintegrable sealing composition, e.g. by controlling its composition.

By some embodiments, the onset of disintegration of the sealing zones is at least about 5 minutes after completion of deployment of the device into its expanded state. By other embodiments, the onset of disintegration of the sealing zones is at least about 10 minutes after completion of deployment of the device into its expanded state.

According to some embodiments, a plurality of the sealing zones comprises the water-disintegrable sealing composition. In such a case, the sealing zones can all comprise the same water-disintegrable sealing composition, or the sealing zones can comprise different water-disintegrable sealing composition, thereby causing gradual disintegration of the device.

According to some other embodiments, a region is defined in the sealing zone that comprises said water-disintegrable sealing composition, and each sealing zone can comprise one or more such regions. The regions can comprise the same or different water-disintegrable sealing composition.

By some embodiments, the water-disintegrable sealing composition connecting the sealing zones to one another in the connecting zones may be the same as the water-disintegrable sealing composition within the sealing zones. By other embodiments, the water-disintegrable sealing composition connecting the sealing zones to one another in the connecting zones is different from the water-disintegrable sealing composition within the sealing zones.

According to some embodiments, the water-disintegrable sealing composition comprises at least one first hydrophilic material and at least one second hydrophilic material, differing in their hydrophilicity and water solubility. According to some embodiments, the first hydrophilic material is less hydrophilic than the second hydrophilic material, and the first hydrophilic material is also less water-soluble than the second hydrophilic material.

The difference in hydrophilicity and solubility can be used to control the timing and rate of disintegration of the water-disintegrable sealing composition.

The term hydrophilic material means to denote a compound or a composition that has high affinity to water. In the water-disintegrable sealing composition, the difference in hydrophilicity allows the water-disintegrable sealing composition to function both as a bonding composition between the two layers of water-insoluble deformable film in the sealing zone, as well as provide controlled disintegration of the sealing zones.

The first hydrophilic material is both less hydrophilic and less soluble than the second hydrophilic material. The first hydrophilic material is selected such that, due to its lower hydrophilicity, it is chemically and/or thermodynamically compatible with the water-insoluble deformable film. Such compatibility permits bonding of the peripheral segments of the water-insoluble deformable films one to the other by the water-disintegrable sealing composition. The first hydrophilic material is selected to permit at least partial dissolution or physical integration into the water-insoluble deformable film portions during formation of the sealing zones (e.g. by thermal welding, ultrasonic welding, solvent bonding, etc.), thereby permitting a continuous interface between the water-insoluble deformable film portions and the water-disintegrable sealing composition. The compatibility thus enables forming closed compartments, having the sealing zones that maintain their mechanical integrity during the volume change of the compartment when transitioning between the collapsed and expanded states.

The first hydrophilic material typically undergoes chemical or physical disintegration when exposed to proper pH conditions in the intestine, typically at pH of about 6-7. As the first hydrophilic material is selected to react when exposed to defined conditions in the intestine, such selection provides further control over the disintegration of the device after ingestion, preventing undesired disintegration when the device passes through the stomach, however permitting disintegration at pH conditions in the intestine.

However, due to its lower solubility and the limited exposure to water resulting from the structure of the sealing zone (i.e. the limited surface area of the water-disintegrable sealing composition layer exposed to water), it requires suitable environment for disintegration.

For obtaining such suitable disintegration environment, the water-disintegrable sealing composition comprises the second hydrophilic material, which has a higher hydrophilicity. The second hydrophilic material functions to quickly absorb and capture the water diffusing into the sealing zone, thereby forming an environment that supports the solubilization of the first hydrophilic material to permit disintegration of the sealing zone and the loss of integrity (i.e. breakdown or disintegration) of the device. Being of higher hydrophilicity, the second hydrophilic material permits also controlled delivery of water into the sealing zone after deployment of the device, thereby controlling the exposure of the first hydrophilic material to water and further controlling the onset of its disintegration.

The combination of the first and second hydrophilic materials permits high controllability over the disintegration rate of the sealing zones: as the water-insoluble films sandwich between them the water-disintegrable sealing composition, and as water diffusivity through the films is limited, relatively small amount of water is capable of penetrating into the sealing zones. The second hydrophilic material permits quick absorption and capture of the water diffusing into the sealing zone, thereby enabling to control the exposure of the first hydrophilic material to water for the solubilization thereof. As the first hydrophilic material functions to form the bond between the two water-insoluble films, increased absorption of water by the second hydrophilic material provides the first hydrophilic material with proper conditions for its solubilization, thereby causing disintegration of the sealing zones and loss of integrity of the compartments and/or device. By balancing and selecting the first and second hydrophilic material, different disintegration onset and disintegration rates can be obtained.

According to some embodiments, the first hydrophilic material can be selected from hypromellose phthalate, hypromellose acetate succinate, methacrylic acid-methyl methacrylate copolymer (e.g. Eudragit L, Eudragit S), methacrylic acid-ethyl acrylate copolymer (e.g. Kollicoat MAE), Cellulose acetate phthalate, and others, as well as combinations thereof.

According to some embodiments, the second hydrophilic material can be selected from hydroxypropyl methylcellulose (HPMC), Hydroxypropyl cellulose (Klucel), cellulose ether (e.g. metolose), polyvinylpyrrolidone and derivatives thereof (e.g. povidone, kollidone, plasdone) polyvinyl alcohol, and others, as well as combinations thereof.

According to some embodiments, the second hydrophilic material is a super-hydrophilic material.

By some embodiments, the amount of the first hydrophilic material in the water-disintegrable sealing composition is larger than the amount of the second hydrophilic material. According to other embodiments, the weight ratio between the first hydrophilic material and the second hydrophilic material is between about 4:1 and about 3:2.

The devices of this disclosure comprise closed compartments formed out of water-insoluble films, with the sealing zones defining a perimetric border of the compartments. According to some embodiments, the compartments are formed by two substantially continuous deformable films, attached to one another to form, the compartments being connected to one another by the connecting zones. In other words, by such embodiments, the device is formed out of two continuous films, that are divided by the sealing zones to form two or more compartments, such that adjacent compartments are spaced-apart by the connecting zones.

By some embodiments, the connecting zones comprise the sealing zones, i.e. the sealing zones form an integral part of the connecting zones. By other embodiments, the connecting zones are constituted by two or more sealing zones bonded to one another by water-disintegrable sealing composition.

According to other embodiments, wherein each compartment is formed out of two distinct deformable film portions, the connecting zones being constituted by two or more sealing zones of adjacent compartments that are bonded to one another; i.e. in such embodiments, the connecting zones consist of stacked connecting zones, forming a layered structure of alternating layers of the water-insoluble deformable films and water-disintegrable sealing composition layers.

The compartments are formed, as noted, out of water-insoluble deformable films, and define a space that holds therein one or more gel forming materials. The term continuous deformable film means to denote a monolithic film, a film constructed as one seamless unit. The term deformable film portions means to denote film segments, that, once attached to one another, form a continuous structure of the device.

The film is typically made of one or more substantially water-insoluble polymeric materials.

In order to permit water to enter the compartments and activate the gel forming material to induce its expansion, and hence deployment of the device, the compartments have one or more liquid-permeable sections. In some embodiments, save said one or more sections of liquid-permeable material, the deformable film is made of non-permeable material. In other words, the deformable film can be made out of two or more different materials, integrally formed one with the other, one material being liquid-permeable and the other material being non-permeable to liquid.

By some embodiments, said sections of the deformable film differ one from the other in their liquid permeability. For example, the sections can differ in their composition, porosity, thickness, size and density of perforations, etc.

The liquid-permeable sections are made of liquid-permeable material. Within the context of the present disclosure, the term liquid-permeable material is meant to denote a material (compound or composition of matter) that permits diffusion or passage of liquid therethrough. For example, the liquid permeable material may be perforated or porous. According to some embodiments, the liquid-permeable material may comprise one or more compounds selected from hypromellose phthalate, cellulose acetate phthalate, hypromellose acetate succinate, cellulose acetate, cellulose acetate butyrate, ethylcellulose, polymethylmethacrylate, polyethylacrylate, polyvinyl acrylate phthalate, polyvinyl acetate, shellac, carboxymethylethylcellulose (CMEC), and any combinations thereof.

The liquid-permeable material may, according to some embodiments, further comprise at least one binder, plasticizer, pore-former, emulsifier, film-former, and any combinations thereof.

For administration, the device is in a collapsed state, i.e. having a compact configuration with a given initial volume. Once administered and exposed to the proper conditions, as will be further discussed below, penetration of liquid through the liquid-permeable sections of the film causes the gel forming material to swell, thereby increasing in volume and irreversibly deploying the device into an expanded state.

According to some embodiments, the device is designed to deliver one or more active agents to a target site in the intestine by direct contact of the active agent or a composition comprising it with the intestinal tissue. For this purpose, the device can comprise, by some embodiments, at least one active agent carrying element, attached to at least a portion of an external surface of at least one of the compartments, such that expansion of the device from the collapsed state to the expanded state causes expansion of the compartment to drive the active agent carrying element towards a tissue and hold the active agent carrying element against the tissue for a predetermined period of time to allow delivery of the active agent to the tissue.

The active agent carrying element is meant to denote any structure or composition that can contain the active agent and permit its release once in contact with the intestinal tissue. The active agent carrying element can be, for example, a layer coating at least a portion of the external surface of the compartment, and constituted by the active agent, or comprising a carrying matrix in which the active agent is dispersed or embedded. By another example, the active agent carrying element can be a solid composition, e.g. a tablet or a pill made of (or containing) the active agent. By some other examples, the active agent carrying element can be in the form of a gel or gel-forming matrix containing the active agent. By a further example, the active agent carrying element can be in the form of a pressure-sensitive element that is designed for rupturing once pressure is applied thereonto after expansion of the compartment as the reservoir is held against the tissue (e.g. the reservoir can be in the form of a casing/envelope that holds therein the active agent or a composition thereof, the casing/envelope having at least one wall portion that is configured for rupturing under a predefined applied pressure).

According to some embodiments, the device is designed to deliver at least one tissue attachable element to a target site in the intestine. Thus, by some embodiments, the device comprises at least one tissue-attachable element attached to at least a portion of an external surface of at least one of the compartments, such that expansion of the device from the collapsed state to the expanded state causes expansion of the compartment to drive the tissue-attachable element towards a tissue of the gastrointestinal tract, for attaching at least part of the tissue-attachable element to the tissue.

The tissue-attachable element is meant to denote an element which is capable of self-attaching to a tissue, typically a mucosal epithelial tissue. The attachment of the tissue-attachable element to the tissue can be by mechanical means, e.g. microneedles or microhooks, which can temporarily anchor into the tissue upon application of pressure by the self-expandable core. Alternatively, the tissue-attachable element can comprise or by constituted by a mucoadhesive material.

In the context of the present disclosure, the term tissue refers to any organ surface or biological membrane that may typically be coated by mucus or mucous membrane (e.g. the mucosal epithelial tissue), typically the tissue is gastric tissue or intestinal tissue.

According to some embodiments, the compartments can be identical to one another or different from one another (e.g. in any one of size, shape, active agent, type, size or shape of active agent carrying element and/or tissue-attachable element, disintegration rate, etc.). According to some embodiments, the device can comprise one or more compartments that carry an active agent carrying element or a tissue-attachable element, while other compartments of the device may be devoid of such.

By varying the size, geometry, number, etc. of the compartments, as well as the type of gel forming material and/or active agent carrying element and/or the tissue-attachable element, different expansion rates, expanded shapes and/or targeted delivery can be obtained. Varying the size, number, geometry, etc. of the compartments can also be utilized to obtain symmetrical or non-symmetrical expanded shape of the device. Varying the properties of the gel-forming material and/or the deformable film can also permit controlling the force applied by the device, in its expanded state, on the tissue for contacting the active agent carrying element or the tissue-attachable element therewith. Further, by folding the device to obtain the collapsed state and/or controlling the number and/or position of the liquid-permeable sections of the deformable film, control over the exposure of the gel-forming material to the liquid can be obtained, thus controlling the overall expansion rate of the device.

According to some embodiments, the tissue-attachable element is a tissue-attachable layer. By some embodiments, the tissue-attachable layer is a mucoadhesive layer that comprises at least one mucoadhesive material.

The term mucoadhesive (or any lingual variation thereof) is meant to denote a compound or composition of matter that is capable of adhering to a tissue, typically via the mucus or mucous membrane. The mucoadhesive material typically interacts with mucus present on or secreted by the tissue, e.g. one or more interactions such as electrostatic interaction, physical entanglement or interpenetration, diffusion, adsorption, mechanical interlocking, etc.

The mucoadhesive material is typically a polymer, that can be natural, semi-synthetic or synthetic. Non-limiting examples of mucoadhesives are tragacanth, sodium alginate, guar gum, xanthan gum, karaya gum, gellan gum, carrageenan, soluble starches, gelatin, chitosan, cellulose derivatives (such as methylcellulose, ethylcellulose, hydroxylethylcellulose, hydroxylpropylcellulose, hydroxypropylmethylcellulose, sodium carboxymethylcellulose (NaCMC)), polyacrylic acid (PAA) polymers (such as carbomers, polycarbophil), polyhydroxyl ethylmethylacrylate, polyethyleneoxide (PEO, typically high molecular weight PEO), polyvinylpyrrolidone (PVP), polyvinyl alcohol (PVA), lectins, pectin, thiolated polymers (for example chitosan-iminothiolane), poly(acrylic acid)-cysteine, poly(acrylic acid)-homocysteine, polyethylene glycol, chitosan-thioglycolic acid, chitosan-thioethylamidine, alginate-cysteine, poly(methacrylic acid)-cysteine, sodium carboxymethylcellulose-cysteine, and others.

In some additional embodiments, the device may include more than one type of a tissue-attachable layer. Different regions of the external surface of the compartment and/or different compartments may be coated by different types of tissue-attachable layers. These different types of tissue-attachable layers may include different types of active agents.

In some additional embodiments, the tissue-facing surface of the tissue-attachable layer (or portions thereof) may be covered by a non-mucoadhesive material (layer) that can be configured to disintegrate to permit exposure of the mucoadhesive layer during or after expansion of the device.

The device can function to deliver at least one active agent to a target site through and/or across the tissue. Thus, according to some embodiments, the tissue-attachable element comprises or carries one or more active agents. Depending on the type of active agent to be delivered (e.g. polarity, hydrophobicity/hydrophilicity, size, etc.), the active agent can be contained within or onto the surface (external surface that is tissue facing, and/or inward-facing surface that is gel forming material-facing surface) of tissue-attachable layer.

In some embodiments, when the tissue-attachable element is a mucoadhesive layer, the active agent is embedded within the mucoadhesive material. For example, the active agent can be dispersed or dissolved in the mucoadhesive material.

In other embodiments, the active agent can be encapsulated within various microparticulate or nanoparticulate structures, such as liposomes, microparticles, microcapsules, nanoparticles, or nanocapsules, the structures being distributed within the mucoadhesive material.

By another embodiment, the active material coats at least a surface portion of the tissue-attachable element; for example, the active material can coat at least one portion of one or both of a tissue-facing surface and a deformable film-facing surface of the tissue-attachable element.

According to further embodiments, the active agent can be linked to the mucoadhesive material by one or more linker moieties, that are susceptible to defined biological conditions, as to permit release of the active agent therefrom once exposed to such conditions.

In some embodiments, when the tissue-attachable element (e.g. a tissue-attachable layer) comprises microneedles, the active agent can be embedded within the microneedles, for example within a polymer from which the microneedles are construed.

The active agent is typically a pharmaceutical active agent. The term pharmaceutical active agent means to denote molecules, compounds or compositions that are safe and effective for pharmaceutical use in a subject, typically mammals, and that possess the desired biological activity. The active agent can be selected, for example, from antibiotics, proteins, peptides, polypeptides, lipids, nucleic acids, hormones, steroids, antibodies, vitamins, anti-inflammatories, antihistamines, antiemetics, analgesics, chemotherapeutic agents, prophylactic agents, clotting factors, radiopharmaceuticals, contrasting agents, electrolytes, nutraceuticals, small molecules (of a molecular weight of less than about 1,000 Da or less than about 500 Da), etc.

In other embodiments, the active agent can be one or more microorganisms (e.g. intestine-friendly bacteria) and/or viruses.

In other embodiments, the active agent can be a nutraceutical compound.

The active agent can be in the form of a salt, acid-addition salt, free base, hydrate, solvate, or prodrug.

The active agent can be suitable for administration to humans. In other embodiments, the active agent can be a veterinary active agent.

The active agent, when present in the tissue-attachable element or when an active agent carrying element is present in the device, is typically in a therapeutically effective amount. The effective amount for purposes herein may be determined by such considerations as known in the art. The amount must be effective to achieve the desired therapeutic effect, depending, inter alia, on the type and severity of the disease to be treated and the treatment regime. The effective amount is typically determined in appropriately designed clinical trials (dose range studies) and the person versed in the art will know how to properly conduct such trials in order to determine the effective amount. As generally known, the effective amount depends on a variety of factors including the affinity of the ligand to the receptor, its distribution profile within the body, a variety of pharmacological parameters such as half-life in the body, on undesired side effects, if any, on factors such as age and gender, and others.

The pharmaceutical active agent can be selected to induce at least one effect, e.g. therapeutic effect, which is capable of inducing, enhancing, arresting or diminishing at least one effect, by way of treatment or prevention of unwanted conditions or diseases in a subject. The at least one agent (substance, molecule, element, compound, entity, or a combination thereof) may be selected amongst therapeutic agents, i.e. agents capable of inducing or modulating a therapeutic effect when administered in a therapeutically effective amount.

In other embodiments, the active agent can be a diagnostic agent, i.e. an agent that permits diagnosis of one or more conditions or disorders. A diagnostically effective amount refers to an amount of the diagnostic agent, which allows for efficient molecular imaging depending on the type of the imaging technique used (e.g. PET, SPECT, etc.), the acquisition parameters of the specific imaging technique used, the area of the body scanned, the physical condition of the subject, the purpose of the test or any other factors which are apparent to the person skilled in art.

In some embodiments, the device can comprise at least one additional active substance, being different from said at least one active agent. The additional active substance can have similar pharmaceutical activity as the active agent, or have a different pharmaceutical activity to that of the active agent.

By some embodiments, the active agent carrying element or the tissue-attachable element comprises the additional active substance. In such embodiments, the active agent and the additional active substance can have a co-therapeutic effect, i.e. additional or synergistic. For example, the additional active substance can function to increase permeation or bioavailability of the active agent, or can increase or enhance the therapeutic effect or bioactivity of the active agent.

By other embodiments, the additional active substance can be contained within the compartments, e.g. associated with the deformable film, associated with the gel-forming material, or mixed (or dispersed) into the gel-forming material. In such cases, the additional active substance may be selected to have an immediate or a short-term therapeutic effect, while the active agent can be selected to have a prolonged or sustained therapeutic effect.

In other embodiments, the active agent and the additional active substance can be selected from agents having similar or identical therapeutic effects. For example, the active agent and the additional active substance can be the same, however one being contained within the active agent carrying element or the tissue-attachable element and the other within the compartment, respectively.

According to other embodiments, the device comprises both an active agent carrying element and a tissue-attachable element, containing the same active agent or different active agents. Such an arrangement can be utilized to divide the required dose of the agent, such that a portion of the agent is released immediately upon expansion and contact of the active agent carrying element with the tissue, and the rest will be slowly absorbed from the tissue-attachable element once attached to the tissue. By another example, the active agent and the additional active substance can have similar effects, with the additional active substance having an immediate effect and the active agent having a prolonged or sustained effect.

In other words, the device can be used to simultaneously administer two or more pharmaceutically active agents, i.e. administered concurrently one after the other. Simultaneous administration may permit one agent in the combination to be administered within a certain time period (e.g. 5 minutes, 10 minutes or even a few hours) after the other, provided that the circulatory half-life concentration of the first administered agent in a combination is concurrently present in therapeutically effective amounts with the other agent administered thereafter. The time delay between administration of the agents may vary depending on the exact nature of the agents, the interaction between the individual agents, their respective half-lives, and on such other factors as easily recognized by the versed artesian.

In a further example, the active agent and the additional substance can have different effects, and the device is designed for sequential administration; meaning that a time difference exists between administering one agent and the other. Such time different may be short or may be significant, i.e. the first administered agent may no longer be present (or is present in subclinical amounts) in the bloodstream in a therapeutically effective amount when the second (or subsequent) agent is administered.

In some embodiments, the tissue-attachable element may further comprise one or more additional components. Such additional components may be, for example, emulsifying agents (surfactants), such as poloxamers or carbomers; stabilizing agents, such as carboxymethylcellulose; suspending agents, such as cellulose, talc; acidifying agents, such as citric acid or ascorbic acid; viscosity increasing agents, such as carbopol, polyethylene oxide; effervescent agents, such as sodium bicarbonate, ammonium carbonate; solubilizing agents, such as lecithin; antimicrobial preservatives, such as sorbic acid, potassium sorbate; antioxidants, such as alpha tocopherol, butylhydroxy anisole; release modifying agents, such as Tween 80, sodium lauryl sulfate; coating agents, such as ethylcellulose, cellulose acetate; binders, such as hydroxypropyl cellulose, polyvinylpyrrolidone; stiffening agents, such as stearic acid, wax; plasticizers, such as diethyl phthalate, triethyl citrate; and others.

As noted, the device undergoes a transformation from a collapsed state to an expanded state as a result of an expansion of a gel forming material that is contained within the compartments.

The term gel-forming material is meant to denote a compound or a composition that is capable of absorbing liquid(s), thereby forming a three-dimensional voluminous network of molecules. The gel-forming material may form a physical gel (i.e. a gel in which molecules are held in the network by physical forces) or a chemical gel (i.e. a gel in which molecules are chemically bonded one to the other to form the network structure).

By some embodiments, the gel-forming material comprises one or more gel forming compounds. By other embodiments, the gel-forming material comprises one or more additives.

According to some embodiments, the gel-forming material comprises one or more polymers. According to other embodiments, the gel-forming material may be charged or neutral.

According to some other embodiments, the gel-forming material is cross-linked or is cross-linkable. Without wishing to be bound by theory, the molecular weight of the gel forming material and the degree of cross-linking have significant impact on the gel's consistency (for example, hardness or rigidity), as well as on its rheological properties (e.g. viscosity). Hence, various molecular weights and cross-linking degrees are some of the parameters that can be used to control the behavior of the zones, thereby controlling the rate of deployment and/or the expanded size of the device.

In some embodiments, the gel-forming material may be selected from gelatin, alginate, chitosan, dextran, collagen, hyaluronic-acid, polyglutamic-acid, elastin, calcium polycarbophil, acrylamides, styrene maleic anhydride, polyethylene oxide, polyacrylic-acid, polyethylene glycol, carboxy methyl cellulose, polyvinyl pyrrolidone, sodium polyacrylate, hydroxypropyl methylcellulose or any combination or composition thereof.

By some embodiments, the gel-forming material is a composition comprising at least one charged gel-forming compound and at least one compound having an opposite charge, constructing a PEC (Poly Electrolyte Complex) formation upon liquid adsorption. In some embodiments, said at least one charged gel-forming compound is selected from polyvinyl acetate diethyl amino acetate (AEA), poly-lysine, chitosan, polymethacrylate (Eudragit E), poly-arginine. In other embodiments, said opposite charged compound is selected from gelatin, hyaluronic-acid, sodium polyacrylate, heparin, polyacrylic acid (Carbomer), alginate, pectin, carboxymethylcellulose.

In some other embodiments, the gel-forming material is at least one super absorbent polymer (SAP). The term super absorbent polymer refers to a polymer (typically cross-linked) or a polymer composition, that can absorb and retain large quantities of liquids, such as water (or liquids containing water), relative to the dry mass of the polymer. Non-limiting examples of SAP are polyethylene glycol (PEG), polyglutamic acid (PGA), polyacrylamide, alginic-acid, dextran, polyacrylic acid, carboxymethylcellulose (CMC), pullulan, starch, and any combinations thereof.

In some other embodiments, the gel-forming material has a swelling ratio of about 10 to 100 times-fold (w/w) (under conditions of: gastrointestinal tract pH at 37° C. for 1 hour).

The term swelling ratio denotes the expansion extent of the gel-forming material between the state prior to adsorbing liquid (i.e. dry or semi dry form) and after adsorbing the maximal possible amount of liquid. The swelling ratio is weight-based determined and calculated according to the following equation:

$$[ ( wet weight ) - ( dry weight ) ] / [ ( dry weight ) ] .$$

The gel-forming material can be in the form of a gel film (i.e. a substantially continuous layer of gel). According to some embodiments, the gel-forming material is in the form of gel particles. By other embodiments, the gel forming particles are in the form of a gel film, the gel particles embedded within a matrix forming a film. In some embodiments, the gel forming particles in the gel film are arranged substantially as a monolayer of gel particles.

According to yet other embodiments, the gel-forming material can be in the form of a powder. In some embodiments when the gel-forming material is in the form of gel particles, the average diameter of the particles of the gel-forming material can range between about 100 μm and about 300 μm.

According to some embodiments, each of the compartments may comprise a different gel-forming material. In some other embodiments, all the compartments can comprise the same gel-forming material.

As the compartments are made of deformable film portions or a continuous deformable film, its folding in various manners is enabled in order to reduce the size of the device and obtain the collapsed state. Thus, the device can be easily administered to a patient in need thereof and into the target organ or organ cavity. In some embodiments, the device is encapsulated in its collapsed state in a self-administrable capsule, to swallowed by the patient.

In some embodiments, when in the collapsed state, the device is folded in a primary folded configuration and is configured to undergo unfolding during transition from the collapsed state to the expanded state. In other words, the device may be folded to assume its collapsed state, having an overall reduced size or overall reduced volume. Once liquid permeates through the liquid-permeating sections of the film, the gel-forming material starts to swell and increase in volume. This, in turn, applies force onto the deformable film, and due to its flexibility and/or deformability, the film is simultaneously unfolded and expanded to assume the device's expanded state.

According to some embodiments, in order to further assist in unfolding, the device comprises at least one deployment unit, that is positioned between folded sections of the device when in said primary folded configuration. The deployment unit is configured to expand upon contact with liquid to assist unfolding of the device.

According to some embodiments, the deployment unit comprises a liquid-permeable casing and at least one gas forming material contained therein. The liquid-permeable casing of the deployment unit permits permeation of liquid thereinto, thereby reacting with the gas forming material to obtain rapid formation of gaseous species. The liquid-permeable casing is typically formed as a closed structure; hence, as the gas expands the casing rapidly, a gas-filled “balloon” is obtained, that pushes onto the folds of the device. Together with the gel forming material undergoing expansion, the inflation of the deployment unit assists in unfolding of the device into its unfolded configuration.

The gas forming material is a compound or a composition that undergoes a chemical reaction in the present of water (either a reaction of the compound with water, or a reaction between components of the composition that is facilitated by exposure to water), with at least some of the reaction products being in gaseous form. An example of such a material can be a solid composition of acid and base (e.g. a composition that contains citric acid and a metal bicarbonate), that once exposed to water—can dissolve and react with each other to produce CO₂ as one of the reaction products.

By some embodiments, when the device is in its primary folded configuration, the device is enveloped by an enteric envelope. The enteric envelope is configured to hold the device in its primary folded configuration, for example by forming the enteric envelope to have dimensions similar to those of the device when in its primary folded configuration. By some embodiments, the enteric envelope is tightly fit over the device in its primary folded configuration, substantially without having spaces formed between the enteric envelope and the device.

The term enteric material means to denote a compound or composition that is configured to be disintegrated or solubilized by a liquid only at a defined pH range. For example, and preferably, the enteric material is stable (that is, maintain their physical and chemical structure) when exposed to acidic environments (for example in the stomach), and are solubilized by more alkaline liquids (such as those in the intestine).

The enteric envelope functions to maintain the device in its folded configuration after intake, until reaching proper conditions (for example, proper pH) within the GI tract that degrade the enteric envelope to permit exposure of the device and its unfolding and expansion.

In order to obtain further compactization in the collapsed state, the device, by some embodiments, has a secondary, rolled configuration, whereby the device that is folded to its primary folded configuration is further rolled about an axis thereof, and is configured to simultaneously undergo unrolling and unfolding during transition from the collapsed state to the expanded state.

According to some embodiments, when in its secondary folded configuration, the device can be enveloped in an enteric envelope. Alternatively, the device can be enveloped by a first enteric envelope when in its primary folded configuration, and by a second enteric envelope when in its secondary folded configuration. In other words, the device can be enveloped by a first enteric envelope after folding it into the primary folded configuration, then folded or rolled into the secondary folded configuration, and enveloped by the second enteric envelope to maintain the device in its secondary folded configuration.

According to other embodiments, the device may first be rolled and then folded in order to assume the collapsed state.

In order to prevent undesired or pre-mature deployment of the device, and/or in order to permit delivery of the device into the proper target site, the device may comprise a biodegradable shell, encapsulating the device in its collapsed state. The biodegradable shell is thus selected to be degraded upon exposure to the proper biological conditions (e.g. pH, presence of certain chemical compounds, etc.).

As the device is typically intended for oral administration and designed to be deployed in the intestine, the biodegradable shell can be made of or coated by an enteric coating.

In some embodiments, the device is an ingestible device, intended, for example, to be deployed in the stomach or in the intestine. Thus, such devices are typically encapsulated within a shell degradable in the gastrointestinal track, encapsulating the device in its collapsed state. For deployment in the intestine, the shell can be designed or selected such as to provide safe first passage through the stomach and biodegrade only when exposed to defined conditions in the intestine. By some embodiments, the gastrointestinal track degradable shell is designed to disintegrate as a function of the pH of the environment, thereby deploying in the desired part of the intestine. For example, different parts of the gastrointestinal tract are known to have different pH values—while the stomach typically has a pH 1.5-3.5, the pH in the duodenum is typically 6, and gradually increasing to about 7.4 in the small intestine (until reaching the terminal ileum). The pH drops to 5.7 in the caecum, but again gradually increases, reaching pH 6.7 in the rectum. Hence, by utilizing a gastrointestinal track degradable shell that degrades at defined pH values (or range of values), deployment of the device at the desired part of the gastrointestinal track can be obtained.

The biodegradable shell can be, for example, a capsule.

The device, by some embodiments, may have two types of shells, one encasing the other. The outer shell in the form of a capsule, to facilitate the swallowing of the device, and is designed to be degradable in the gastric environment. The second, inner, shell may be a coating layer or an encapsulating layer, that coats/encapsulates the device, and is configured to maintain the device in its collapsed state for a predetermined period of time before deployment. For example, the inner shell can be made from an enteric material, thus preventing deployment of the device in the stomach and permitting its deployment only upon entrance to the intestine.

While the device can be used to deliver at least one active agent to a target site, the device can also be devoid of active agents, or comprise only the active substances within the compartment. For example, the device can be utilized to deliver a patch to a wall of a tissue, e.g. to temporarily block passage of substances across the mucosal/epithelial tissue (both from the lumen and/or to the lumen), or to cover perforations or ulcers in the tissue.

As noted, in some embodiments, while the tissue-attachable element may not contain an active agent, the compartments and/or the gel-forming material may comprise an active substance/agent, such as an analgesic or anti-inflammatory, that can be released during the disintegration of the gel-forming material to provide a local desired effect relatively quickly after administration.

In the expanded state, the device may have a circular shape, a polygonal shape, or an irregular shape. In its expanded state, the device may be designed to assume a three-dimensional (3D) shape generally conforming to the shape of at least a section of the lumen or cavity in which it expands (or deploys).

In other embodiments, in the expanded state, the device may have an annular or ring-like shape.

In some other embodiments, the device may be configured to assume a substantially cylindrical shape when in the expanded state, to define a hollow lumen. Such a hollowed cylindrical shape prevents formation of blockage of the luminal organ or organ cavity when the device is in its expanded state, permitting passage of liquids or solids through the organ while the device is deployed therein.

By some embodiments, the device is in the form of a sleeve, the sleeve walls being constituted by the deformable film, wherein the compartments are defined along the circumference of the sleeve, with the tissue-attachable layers coating at least a portion of the external surface of the compartments and facing outwards from the surface of the sleeve. Typically, the compartments are elongated along a longitudinal axis of the sleeve and are arranged parallel to one another along the circumference of the sleeve.

According to some embodiments, in order to prevent adherence of the external layer of the device to itself when the device is in its primary and/or secondary folded configurations, one or more separation layers are placed between one or more of the folds of the device. These one or more separation layers can be attached in one or more attachment locations to the device in order to maintain their position and function when the device is folded into its primary and/or secondary folded configurations. Alternatively, the separation layer(s) can be located in between the folds without anchoring it to the device (for example by placing such a layer over the device before folding, and then folding the device together with the separation layer to form the primary and/or secondary folded configuration).

Various components of the device, e.g. the liquid-permeable sections, the gel-forming material, etc., can be biodegradable. The term biodegradable means to denote any type of decomposition of the device that is caused by exposure to suitable biological conditions after administration and expansion. The term encompasses mechanical breakdown, chemical or physical degradation, chemical or physical decomposition, or any other type of destruction of the integrity of the device during its passage through the gastrointestinal tract for expulsion from the body.

By another aspect, this disclosure provides an ingestible self-expandable device configured for delivery of at least one active agent to a tissue, the device having a collapsed state and an expanded state, and comprising: two or more self-expandable compartments, connected to one another by connecting zones, each compartment being formed out of two, substantially water-insoluble deformable film portions, the two film portions being bonded to each other at peripheral segments of the compartment by at least one water-disintegrable sealing composition to define sealing zones, each sealing zone being a layered structure comprised of said water-insoluble deformable films sandwiching between them at least one water-disintegrable sealing composition layer that bonds said water-insoluble deformable films to one another at the peripheral segments, the two film portions defining between them, at areas enclosed by the peripheral segments, an enclosed space of the compartment, each compartment having one or more liquid-permeable sections and a gel forming material within said enclosed space, the gel forming material being configured for swelling upon contact with liquid, thereby expanding the compartment to irreversibly switch the device from the collapsed state to the expanded state; the water-disintegrable sealing composition provides mechanical stability to the sealing zones during transition of the device from the collapsed state to the expanded state, and having water-solubility configured to provide controlled disintegration of the sealing zones after the device is expanded into the expanded state, to thereby cause loss of integrity of the compartments and/or the device after its deployment, and at least one active agent carrying element attached to at least a portion of an external surface of at least one of the compartments, such that expansion of the device from the collapsed state to the expanded state causes expansion of the compartment to drive the active agent carrying element towards said tissue and hold the active agent carrying element against the tissue for a predefined period of time to permit delivery of the active agent from the active agent carrying element to the tissue.

By another aspect, this disclosure provides an ingestible self-expandable device configured for attaching a tissue-attachable layer to a tissue, the device having a collapsed state and an expanded state, and comprising:
two or more self-expandable compartments, connected to one another by connecting zones, each compartment being formed out of two, substantially water-insoluble deformable film portions, the two film portions being bonded to each other at peripheral segments of the compartment by at least one water-disintegrable sealing composition to define sealing zones, each sealing zone being a layered structure comprised of said water-insoluble deformable films sandwiching between them at least one water-disintegrable sealing composition layer that bonds said water-insoluble deformable films to one another at the peripheral segments,
the two film portions defining between them, at areas enclosed by the peripheral segments, an enclosed space of the compartment, each compartment having one or more liquid-permeable sections and a gel forming material within said enclosed space, the gel forming material being configured for swelling upon contact with liquid, thereby expanding the compartment to irreversibly switch the device from the collapsed state to the expanded state;
the water-disintegrable sealing composition provides mechanical stability to the sealing zones during transition of the device from the collapsed state to the expanded state, and having water-solubility configured to provide controlled disintegration of the sealing zones after the device is expanded into the expanded state, to thereby cause loss of integrity of the compartments and/or the device after its deployment, and
at least one tissue-attachable layer coating at least a portion of an external surface of at least one of the compartments,
such that expansion of the device from the collapsed state to the expanded state causes expansion of the compartment to drive the tissue-attachable layer towards said tissue for attaching at least part of the tissue-attachable layer to the tissue.

Another aspect of this disclosure provides an ingestible self-expandable arrangement that comprises an ingestible self-expandable device having a collapsed state and an expanded state, and at least one deployment unit having a non-inflated state and an inflated state. The ingestible self-expandable device comprises two or more self-expandable compartments, connected to one another by connecting zones; each compartment being formed out of two, substantially water-insoluble deformable film portions, the two film portions being bonded to each other at peripheral segments of the compartment by at least one water-disintegrable sealing composition to define sealing zones, each sealing zone being a layered structure comprised of said water-insoluble deformable films sandwiching between them at least one water-disintegrable sealing composition layer that bonds said water-insoluble deformable films to one another at the peripheral segments; the two film portions defining between them, at areas enclosed by the peripheral segments, an enclosed space; each compartment having one or more liquid-permeable sections and a gel forming material within said enclosed space, the gel forming material being configured for swelling upon contact with liquid, thereby expanding the compartment to irreversibly switch the device from the collapsed state to the expanded state. When at the collapsed state, the device is folded in a primary folded configuration, the at least one deployment unit being positioned between folded sections of the device when in said primary folded configuration, and configured to switch from said non-inflated state into said inflated state upon contact with liquid, to permit unfolding of the device.

By another aspect, this disclosure provides an ingestible arrangement for delivery of at least one active agent to a tissue, the arrangement comprising:
an ingestible self-expandable device having a collapsed state and an expanded state, and at least one deployment unit having a non-inflated state and an inflated state,
the ingestible self-expandable device comprising:
two or more self-expandable compartments, connected to one another by connecting zones, each compartment being formed out of two substantially water-insoluble deformable film portions, the two film portions being bonded to each other at peripheral segments of the compartment by at least one water-disintegrable sealing composition to define sealing zones, each sealing zone being a layered structure comprised of said water-insoluble deformable films sandwiching between them at least one water-disintegrable sealing composition layer that bonds said water-insoluble deformable films to one another at the peripheral segments,
the two film portions defining between them, at areas enclosed by the peripheral segments, an enclosed space,
each compartment having one or more liquid-permeable sections and a gel forming material within said enclosed space, the gel forming material being configured for swelling upon contact with liquid, thereby expanding the compartment to irreversibly switch the device from the collapsed state to the expanded state, and
at least one active agent carrying element attached to at least a portion of an external surface of at least one of the compartments,
such that expansion of the device from the collapsed state to the expanded state causes expansion of the compartment to drive the active agent carrying element towards said tissue and hold the active agent carrying element against the tissue for a predefined period of time to permit delivery of the active agent from the active agent carrying element to the tissue; and
when at the collapsed state, the device is folded in a primary folded configuration, the at least one deployment unit being positioned between folded sections of the device when in said primary folded configuration, and configured to switch from said non-inflated state into said inflated state upon contact with liquid, to permit unfolding of the device.

By another aspect, this disclosure provides an ingestible arrangement for attaching a tissue-attachable layer to a tissue, the arrangement comprising:
an ingestible self-expandable device having a collapsed state and an expanded state, and at least one deployment unit having a non-inflated state and an inflated state,
the ingestible self-expandable device comprising:
two or more self-expandable compartments, connected to one another by connecting zones, each compartment being formed out of two substantially water-insoluble deformable film portions, the two film portions being bonded to each other at peripheral segments of the compartment by at least one water-disintegrable sealing composition to define sealing zones, each sealing zone being a layered structure comprised of said water-insoluble deformable films sandwiching between them at least one water-disintegrable sealing composition layer that bonds said water-insoluble deformable films to one another at the peripheral segments,
the two film portions defining between them, at areas enclosed by the peripheral segments, an enclosed space,
each compartment having one or more liquid-permeable sections and a gel forming material within said enclosed space, the gel forming material being configured for swelling upon contact with liquid, thereby expanding the compartment to irreversibly switch the device from the collapsed state to the expanded state, and
a tissue-attachable layer coating at least a portion of an external surface of at least one of the compartments,
such that expansion of the device from the collapsed state to the expanded state causes expansion of the compartment to drive the tissue-attachable layer towards said tissue for attaching at least part of the tissue-attachable layer to the tissue; and
when at the collapsed state, the device is folded in a primary folded configuration, the at least one deployment unit being positioned between folded sections of the device when in said primary folded configuration, and configured to switch from said non-inflated state into said inflated state upon contact with liquid, to permit unfolding of the device.

In other words, an arrangement that includes a self-deployable device and at least one deployment unit is another aspect of this disclosure. The combination of a device and a deployment unit is referred to herein as an ingestible arrangement.

According to some embodiments, the deployment unit comprises a liquid-permeable casing and at least one gas-forming material contained therein, such that contact of liquid with said gas-forming material causes release of gas to inflate the deployment unit.

Typically, as the timing of operation of the gas-forming material is faster than that of the gel-forming material, the deployment unit is first inflated to at least partially unfold the collapsed device, and then the gel-forming material expands in order to further deploy the device into its unfolded configuration.

According to some embodiments, the arrangement comprises a single deployment unit.

According to other embodiments, the arrangement comprises two or more deployment units. The two or more deployment units can be attached to one another or unattached one to the other. The two or more deployment units can be identical one to the other, or can differ from one another by at least one of liquid permeability of the liquid-permeable casing, type of gas forming material, amount of gas forming material, size and/or geometry, etc.

In some embodiments, the at least one deployment unit is attached to the self-expandable device. In other embodiments, the at least one deployment unit is not connected to the self-expandable device in the arrangement.

In some embodiments, the water-disintegrable sealing composition provides mechanical stability to the sealing zones during transition of the device from the collapsed state to the expanded state, and having water-solubility configured to provide controlled disintegration of the sealing zones after the device is expanded into the expanded state, to thereby cause loss of integrity of the compartments and/or the device after its deployment.

By another one of its aspects, the disclosure provides a method for delivery of at least one active agent to a tissue of a subject in need thereof, the method comprising administering to the subject a self-expandable device as disclosed herein, encapsulated in a biodegradable shell.

By a further aspect, there is provided a method of prolonged or sustained delivery of at least one active agent to a tissue of a subject in need thereof, the method comprising administering to the subject a self-expandable device as disclosed herein, encapsulated in a biodegradable shell. Hence, the devices of this disclosure form a reservoir of active agent to be delivered at the target site for a prolonged period of time.

By yet a further aspect, there is provided a method of delivery of at least one active agent to a tissue of a subject in need thereof, the method comprising administering to the subject a self-expandable device as disclosed herein, encapsulated in a biodegradable shell.

By still a further aspect, there is provided a method of attaching, through a mucous membrane, a tissue-attachable layer to a tissue of a subject in need thereof, the method comprising administering to the subject a self-expandable device as disclosed herein, encapsulated in a biodegradable shell.

As used herein, the term about is meant to encompass deviation of ±10% from the specifically mentioned value of a parameter, such as concentration, time, etc.

Whenever a numerical range is indicated herein, it is meant to include any cited numeral (fractional or integral) within the indicated range. The phrases ranging/ranges between a first indicate number and a second indicate number and ranging/ranges from a first indicate number “to” a second indicate number are used herein interchangeably and are meant to include the first and second indicated numbers and all the fractional and integral numerals therebetween.

Unless the context requires otherwise, the term comprise, and variations such as comprises and comprising, will be understood to imply the inclusion of a stated integer or step or group of integers or steps but not the exclusion of any integer or step or group of integers and steps.

Generally it is noted that the term . . . at least one . . . as applied to any component of the device or arrangement of this disclosure should be read to encompass one, two, three, four, or even more different occurrences of said component in the device or arrangement.

It is appreciated that certain features of this disclosure, which are, for clarity, described in the context of separate embodiments, may also be provided in combination in a single embodiment. Conversely, various features of this disclosure, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-1C are schematic representations of an exemplary device according to an embodiment of this disclosure, in which the device is configured as a sleeve: FIG. 1A being a front perspective view, FIG. 1B being a top cross-sectional view across line I-I, FIG. 1C showing the device in an expanded form;

FIGS. 1D-1F are schematic representations of another exemplary device according to an embodiment of this disclosure, in which active agent carrying elements are utilized instead of tissue-attachable elements: FIG. 1D being a front perspective view, FIG. 1E being a top cross-sectional view across line II-II, FIG. 1F showing the device in an expanded form with the active agent carrying elements held be the expanded device against the tissue;

FIG. 2A is a self-expandable compartment seen in isolation, defined between two sealing zones;

FIG. 2B is a top cross-sectional view of a device according to an embodiment of this disclosure, in which the sealing zones are shown;

FIG. 3 is a schematic top view of a device according to another embodiment of this disclosure, in which the compartments are made of distinct portions of deformable film;

FIGS. 4A-4B show schematic representations of a device according to another example of this disclosure in which the compartments are made of distinct portions of deformable film;

FIGS. 4C-4D show schematic representations of a device according to yet another example of this disclosure in which the compartments are made of distinct portions of deformable film;

FIGS. 5A-5C shows the various primary folded configurations of the device of FIG. 1A;

FIGS. 5D-5F show the primary folded device enveloped in an enteric envelope—top view (FIG. 5D), side view (FIG. 5E), and enveloped in a degradable shell (FIG. 5F);

FIGS. 6A-6B show an exemplary primary folded configuration of the device, with the device inclosing a deployment unit in a non-inflated state (FIG. 6A) and an inflated state (FIG. 6B);

FIGS. 6C-6D show another exemplary primary folded configuration of the device, with the device inclosing a deployment unit in a non-inflated state (FIG. 6C) and an inflated state (FIG. 6D).

### DETAILED DESCRIPTION OF EMBODIMENTS

In the following, exemplary devices according to this disclosure will be described. While the specific examples show the device as being substantially symmetric, it is to be understood that the device may also be asymmetric or of any other shape. Further, the elements of the device are shown out of scale for ease of illustration. Further, while the exemplary devices will be shown to have active agent carrying element or tissue-attachable elements, it is to be understood that similar devices that do not include such elements are also included within the scope of the examples.

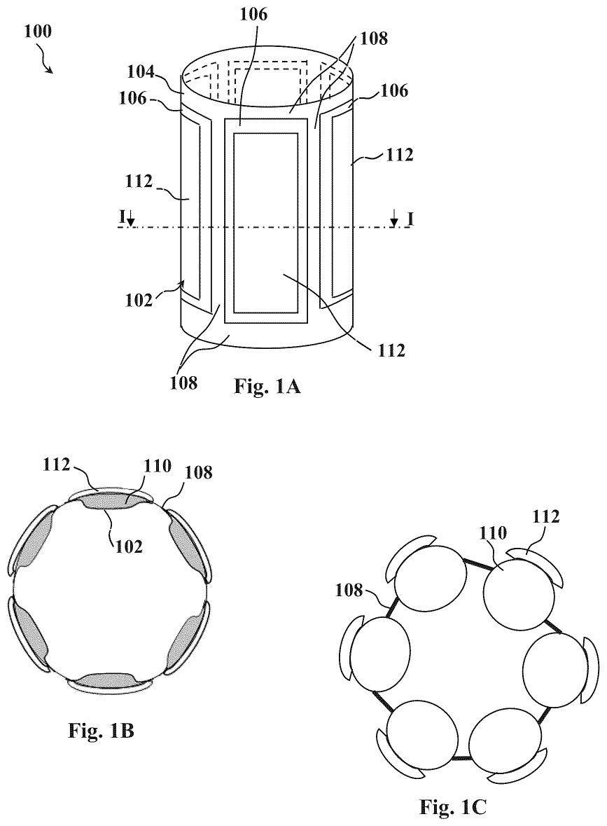

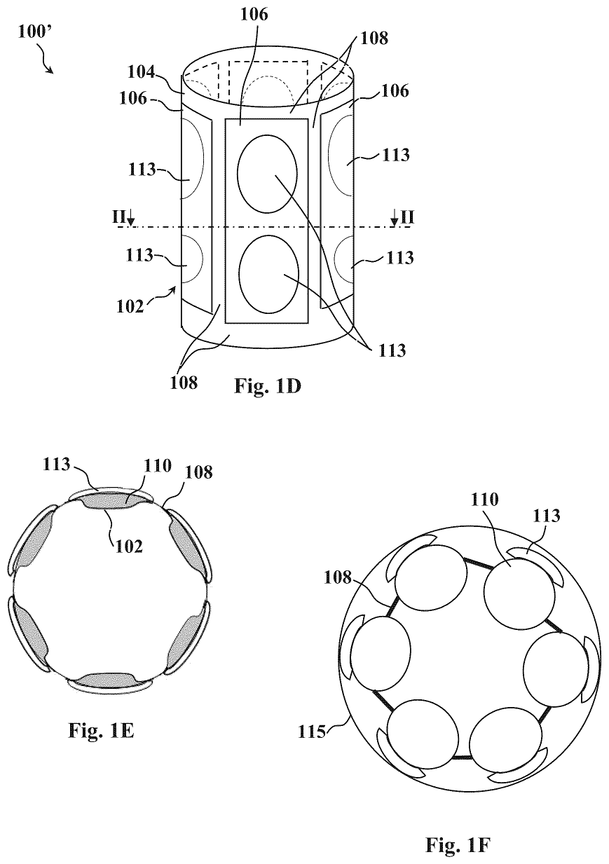

Shown in FIG. 1A is a self-expandable device according to an embodiment of this disclosure, in a collapsed (non-expanded) state. The device 100 of FIGS. 1A-1C has an overall cylindrical sleeve-like shape, and comprises a plurality, in this case six, self-expandable compartments, collectively designated 102, and are formed out of a substantially water-insoluble deformable 2-layered film 104. While in this example six compartments are shown, it is to be understood that any number of compartments can be utilized, e.g. 2, 3, 4, 5, 6, 7, 8 or even more compartments.

In this example, the entire sleeve is constituted by two overlapping layers of substantially water-insoluble deformable film, having sections 106 that are liquid-permeable in order to permit entrance of liquid into the compartments. The deformable films are connected one to the other along the periphery of the compartments by sealing zones 108, which in this example also constitutes the connecting zones that connect between adjacent compartments 102. However, it is also to be understood that the sealing zones can be part of a larger connecting zone that connects between the compartments and spaces the compartments one from the other (not shown).

As the films are attached to one another only in the sealing zones (by a water-disintegrable sealing composition, as explained below), the compartment defines an enclosed space which encloses a gel-forming material 110. The gel-forming material 110 is activated once liquid permeates through sections 106 into the compartments 102, to thereby expand the gel-forming material and switch the device from its collapsed state (shown in FIGS. 1A-1B to its expanded state shown in FIG. 1C. A tissue-attachable element 112 is located on the external surface of each compartment 102 (i.e. the face of the film 104 that faces outwards from the compartment). The tissue-attachable element 112 can be, for example, in the form of a tissue-attachable layer. The tissue-attachable element 112, as noted above, can comprise one or more active agents(s) to be delivered to a tissue. As also noted above, the device can also be devoid of the tissue attachable elements 112, for example as a device that is configured to deliver an active agent that resides on the surface of the compartments, or a device that is configured to temporarily employ pressure onto the tissue (when the device is in its expanded state).

The tissue-attachable element 112 can be provided as patches that are applied (e.g. adhered) onto film 104 over compartments 102. For example, when the tissue-attachable element is a mucoadhesive layer, the patch can comprise the mucoadhesive material, layered upon a backing layer (not shown) that enables adhering or attaching the patch onto the external surface of the compartment and provides support for the mucoadhesive material. Alternatively, the mucoadhesive material can be first applied at specific locations onto film 104, followed by formation of the compartments 102 at locations corresponding to areas of the film containing the mucoadhesive material, thus forming a multilayer film structure.

Seen in FIG. 1C is the device at its expanded form, i.e. after being exposed to liquids that cause swelling of the gel-forming material, thereby expanding the device. Expansion of the device causes the tissue-attachable element to become proximal to the tissue and be held against the tissue due to the increase in volume of the compartments, thereby causing attachment of the tissue-attachable element to the tissue.

Another configuration of the device is shown in FIGS. 1D-1F is a device 100′, similar to the device of FIGS. 1A-1C, however instead of tissue attachable layers 112, device 100′ comprises active agent carrying elements 113.

Seen in FIG. 1D is device 100′, in which active agent carrying elements 113 are attached to self-expandable compartments 102, constructed out of substantially water-insoluble deformable 2-layered film 104. Sections 106 are liquid-permeable, permitting entrance of liquid into compartments 102, to react with gel forming material 110, thereby expanding the compartments 102, and transform the device from its collapsed state (FIG. 1E) to its expanded state (FIG. 1F). As in device 100, in device 100′ the deformable films are connected one to the other along the periphery of the compartments by sealing zones 108.

As seen in FIG. 1F, when the device is transitioned into the expanded state, its volume increases due to the expansion of compartments 102, driving the active agent carrying elements 113 towards the tissue, for example the walls of the intestinal tract 113. The change in volume leads to the active agent carrying elements 113 to become proximal to the tissue 115, until the active agent carrying elements 113 contact(s) the tissue 115. Due to its voluminous state, the active agent carrying elements 113 are held against the tissue as long as device 100 maintains its integrity in the expanded state, to permit delivery of the active agent carried by element 113 to the tissue.

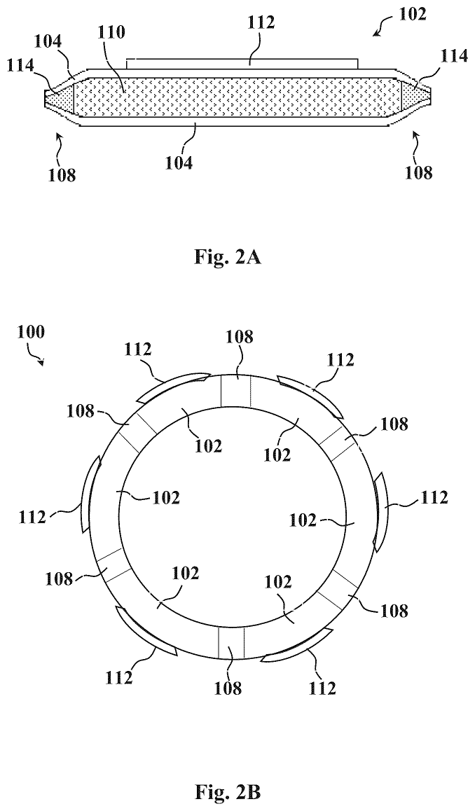

FIG. 2A is a close-up cross-sectional view of one of the compartments (of devices 100 and 100′), showing the structure of the sealing zones. The compartment 102 is defined between portions of films 104, with the portions of films attached to one another at the sealing zones 108 along the peripheries of the compartment. Sealing zones 108 comprise at least one water-disintegrable sealing composition 114 that is positioned as a layer between the peripheral segments of the films 104, thereby forming an enclosed space that defines the interior of the compartment and encloses the gel forming material 110.

The water-disintegrable sealing composition comprises at least one first hydrophilic material and at least one second hydrophilic material, that differ in their hydrophilicity and water solubility.

The first hydrophilic material is both less hydrophilic and less soluble than the second hydrophilic material. The first hydrophilic material is selected such that, due to its lower hydrophilicity, it is chemically and/or thermodynamically compatible with the water-insoluble deformable film. Such compatibility permits bonding of the peripheral segments of the water-insoluble deformable film one to the other by the water-disintegrable sealing composition. The first hydrophilic material is selected to permit at least partial dissolution or physical integration into the water-insoluble deformable film portions during formation of the sealing zones (e.g. by thermal welding, ultrasonic welding, solvent bonding, etc.), thereby permitting a continuous interface between the water-insoluble deformable film portions and the water-disintegrable sealing composition. The compatibility thus enables forming closed compartments, having the sealing zones that maintain their mechanical integrity during the volume change of the compartment when transitioning between the collapsed and expanded states.

The first hydrophilic material typically undergoes chemical or physical disintegration when exposed to proper pH conditions in the intestine, typically at pH of about 6-7. As the first hydrophilic material is selected to react when exposed to defined conditions in the intestine, such selection provides further control over the disintegration of the device after ingestion, preventing undesired disintegration when the device passes through the stomach, however permitting disintegration at pH conditions in the intestine.

However, due to its lower solubility and the limited exposure to water resulting from the structure of the sealing zone (i.e. the limited surface area of the water-disintegrable sealing composition layer exposed to water), it requires suitable environment for disintegration.

In order to obtain such suitable disintegration environment, the water-disintegrable sealing composition comprises the second hydrophilic material, which has a higher hydrophilicity. The second hydrophilic material functions to quickly absorb and capture the water diffusing into the sealing zone, thereby forming an environment that support the solubilization of the first hydrophilic material to permit disintegration of the sealing zone and the loss of integrity of the device. Being of higher hydrophilicity, the second hydrophilic material permits also controlled delivery of water into the sealing zone after deployment, thereby controlling the exposure of the first hydrophilic material to water and further controlling the onset of its disintegration.

The combination of the first and second hydrophilic materials permits high controllability over the disintegration rate of the sealing zones: as the water-insoluble film portions sandwich between them the water-disintegrable sealing composition, and as water diffusivity through the film portions is limited, relatively small amount of water is capable of penetrating into the sealing zones. The second hydrophilic material permits quick absorption and capture of the water diffusing into the sealing zone, thereby enabling to control the exposure of the first hydrophilic material to water for the solubilization thereof. As the first hydrophilic material functions to form the bond between the two water-insoluble film portions, increased absorption of water by the second hydrophilic material provides the first hydrophilic material with proper conditions for its solubilization, thereby causing disintegration of the sealing zones and loss of integrity of the compartments and/or device. By balancing and selecting the first and second hydrophilic material, different disintegration onset and disintegration rates can be obtained.

Seen in FIG. 2B is a cross-section of the device of FIGS. 1A-1B, showing the formation of the compartments between the sealing zones. It is to be understood that the same construction of the compartments and sealing zones apply to the device of FIGS. 1D-IE. As the device has a typical closed form, the layer of water-disintegrable sealing composition 114 is sandwiched between the films 104, significantly limiting the exposure of the water-disintegrable sealing composition to liquid; thus, the second hydrophilic material, that promotes absorption of water into the sealing zone, permits increasing the absorption of water into the sealing zones, to enable dissolution of the first hydrophilic material to disintegrate the sealing zones.

In operation, the device undergoes a series of transformations due to exposure to proper conditions after ingestion. Typically, the device is encased in its folded configurations in a biodegradable shell, e.g. a capsule 118 (as exemplified in FIG. 5F). After the device is administered, e.g. orally, a biodegradable capsule 118 passes the stomach and undergoes disintegration when exposed to suitable conditions in the intestine, thus exposing device 100. As noted, in some configurations, the biodegradable capsule 118 is degraded in the stomach and the device is encapsulated/coated with an enteric layer 122 (FIGS. 5E-5F), that protects the device when passing along the stomach, and is designed to disintegrate when exposed to suitable conditions in the intestine. It is to be noted, however, that in other configurations, the enteric layer 122 may be absent. The enteric layer typically comprises or is formed out of one or more enteric polymers.

Once the capsule 118 (or the enteric layer) is disintegrated, liquid in the intestine permeates the liquid-permeable sections of deformable film 104 of compartment 102, causing expansion of gel-forming material 110 contained therein. Expansion of the gel-forming material causes expansion of the compartment and deformation of film, causing the device to assume its expanded state. In the expanded state, the expansion of the gel-forming material causes application of force onto film 104 against the tissue in the target site, forcing the tissue-attachable element 112 to come into contact with the tissue and adhering thereto (FIG. 1C), or holding the active-agent carrying element 113 against the tissue 115 as long as the device maintains its integrity in the expanded state (FIG. 1F). Hence, the transition of the device from its collapsed state to its expanded state drives the tissue-attachable element 112 (e.g. a mucoadhesive layer) or the active-agent carrying element 113 associated with the compartment towards the tissue and brings it into intimate contact therewith under application of force (applied by the expanding gel-forming material).

After expansion, the water-disintegrable sealing composition in sealing zones 108 absorbs water from the environment, and causes controlled disintegration of the sealing zones 108, leading to loss of integrity (i.e. breakdown or disintegration) of the compartments and to fragmentation of the device, resulting in evacuation thereof from the target site. Once the compartments lose their integrity, the gel forming material 110 is cleared from the target site by the intestinal natural movement, leaving the tissue-attachable element attached or adhered to the tissue. When the tissue-attachable element 112 contains an active agent, the active agent contained within the mucoadhesive element is thus delivered to the tissue during the time period in which the tissue-attachable element remains adhered to the tissue.

As the tissue is routinely shed from the intestinal wall every few hours, shedding of the tissue will result also in detachment and disintegration of the tissue-attachable element, and clearance of the tissue-attachable layer from the intestine.

Alternatively, when the device comprises active-agent carrying elements 113, loss of integrity of the compartments will lead to cease of contact between the active-agent carrying elements 113 and the tissue, followed by evacuation of the device fragments from the target site.

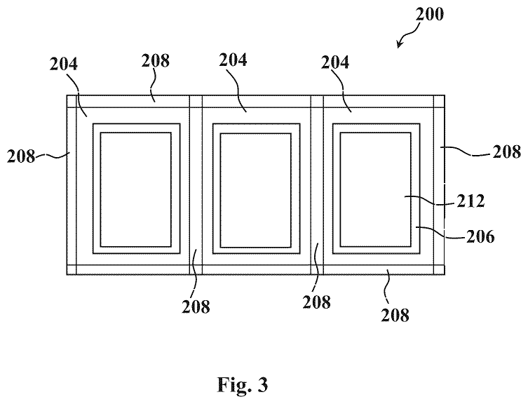

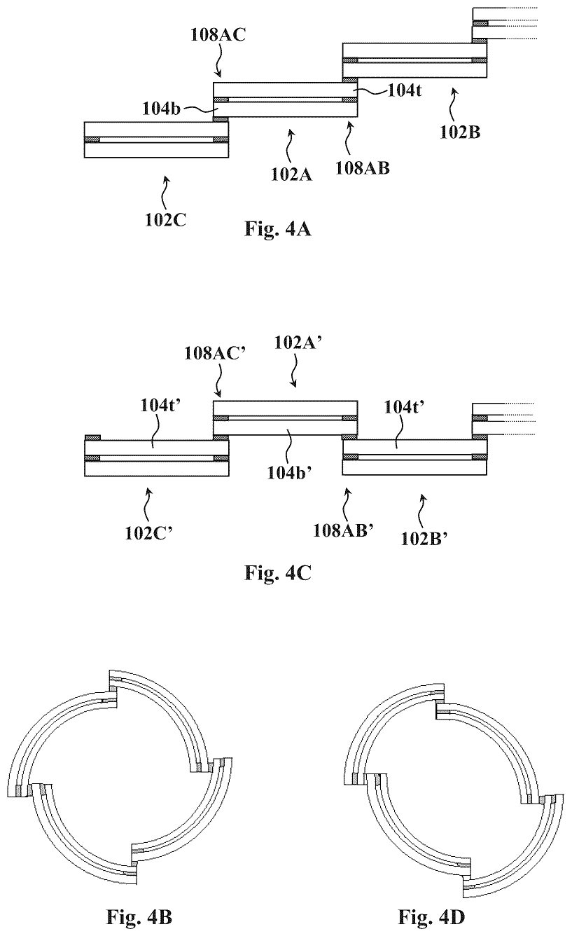

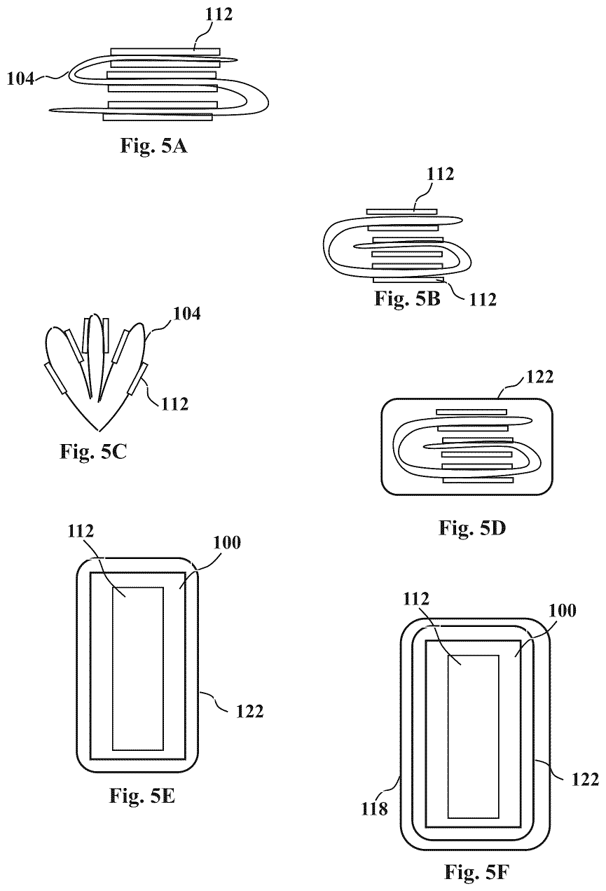

FIG. 3 provides another exemplary construction of a device according to an embodiment of this disclosure. Device 200 is constructed out of individual portions 204 of water-insoluble deformable film, arranged in overlapping pairs to form the compartments, and attached to one another along peripheral portions thereof to form the sealing zones 208 and the space of the compartment that holds the gel-forming material (not marked). Each film portion 204 comprises at least one liquid-permeable section 206, permitting water permeation into the compartment to activate the gel forming material at the target site, to thereby switch the device from its collapsed state to its expanded state (due to the expansion of the gel forming material in the compartments), to permit attachment of the tissue-attachable element 212 to the tissue at the target site. In this example, after expansion, the device will commence disintegration along the peripheral sealing zones 208 once sufficient water is absorbed by the second hydrophilic material to solubilize the first hydrophilic material in the water-disintegrable sealing composition.

It is again noted that instead of tissue-attachable elements 212, active agent carrying elements can be used (not shown), that are held against the tissue to form contact with the tissue (however without adhering thereto) to permit delivery of the active agent to the tissue as long as the device maintains its integrity in its expanded state.

Different configurations of arranging the water-insoluble film portions and constructing the connecting zones in order to obtain a device as shown in FIGS. 4A-4B and 4C-4D. In these examples, the connecting zones are constituted by sealing zones of adjacent compartments that are stacked one over the other.

In the configuration of FIGS. 4A-4B, the compartments are attached to one another such that both films of each compartment are attached to adjacent compartments—for example compartment 102A is attached to two adjacent compartments 102B and 102C; compartment 102B is attached through the top film portion 104t of the sealing zone 108AB, while the other compartment 102C is attached through a bottom film 104b of the sealing zone 108AC of compartment 102A.

In the configuration of FIGS. 4C-4D, the compartments are attached to one another such that only one of the films in each compartment is attached to the adjacent compartments—for example compartment 102A′ is attached to two adjacent compartments 102B′ and 102C′; both compartments 102B′ and 102C′ are attached through their top film portions 104t′ to the bottom film portion 104b′ of compartment 102A′ in the sealing zones 108AB′ and 108AC′, respectively.

The attachment of one compartment to the other is typically made using the water-disintegrable sealing composition.

In the examples of FIGS. 4A-4D, sealing zones of adjacent compartments are stacked one over the other to form the connecting zones. By stacking the sealing zones, the connecting zones comprise a plurality of alternating layers, namely layers of the peripheral segments of the substantially water-insoluble deformable film portions, with layers of the water-disintegrable sealing composition arranged therebetween. Such layered arrangement permits the water-disintegrable sealing composition to function both as a structural component of the device that enables attaching the compartments one to the other during production of the device, as well as provide mechanical integrity during the transition of the device between its folded and un-folded configurations and from the collapsed to the expanded state.

It is of note that the water-disintegrable sealing composition connecting the sealing zones to one another in the connecting zones can be the same as the water-disintegrable sealing composition within the sealing zones, or can be a different water-disintegrable sealing composition. Such possible variation of the water-disintegrable sealing composition permits obtaining different disintegration onsets and/or rates for the connecting zones and the sealing zones; in other words, by selecting different water-disintegrable sealing compositions within the sealing zones and between the sealing zones, gradual disintegration can be obtained—first a breakdown of the device into individual compartments, and then break-down of the individual compartments themselves.

In order to obtain compactization of the device in its collapsed (non-expanded) state, the device may be folded in various folding configurations (i.e. various primary folded configurations, as can be seen in FIGS. 5A-5C) and/or rolling configurations (not shown), to permit its unfolding (and/or unrolling) during transition from the collapsed state to the expanded state. In other words, the device may be folded to assume its collapsed state, having an overall reduced size or overall reduced volume. Once liquid permeates through the liquid-permeable sections of the deformable film, the gel-forming material starts to swell and increase in volume. This, in turn, applies force onto the deformable film, and due to its flexibility and/or deformability, the film is unfolded to assume the device's expanded state.

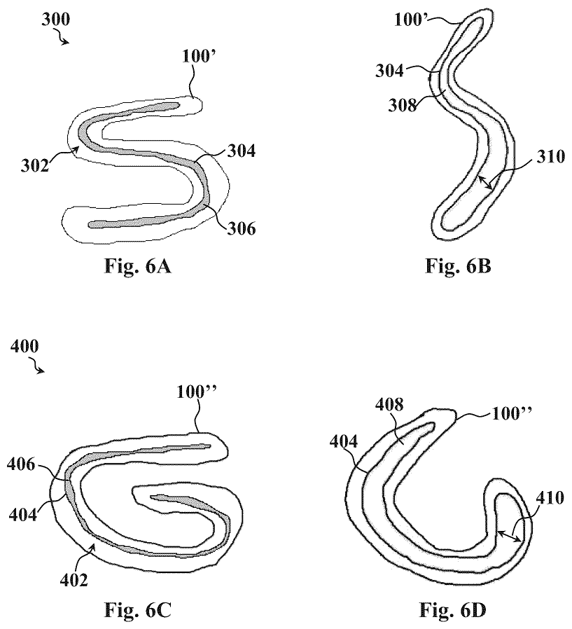

FIGS. 5A-5C show various configurations for folding the device in order to render it with a more compact form for intake. For ease of visualization, only tissue-attachable elements 112 are shown onto the film 104. Further, it is to be understood that active agent carrying elements 113 can be used instead of tissue-attachable elements 112 (not shown in FIGS. 5A-5F). When in the collapsed state, the device can be folded to one of the primary folded configurations shown in FIGS. 5A-6C, and encased in a biodegradable capsule (not shown). After intake and disintegration of the biodegradable capsule, exposure to liquid in the GI track causes expansion of the gel-forming material in the compartments, thereby expanding the compartments and causing at least partial concomitant expansion and unfolding into the expanded state.

Once folded, into the primary folded configuration, the folded device can be enveloped by an enteric envelope 122, as shown for example in FIGS. 5D-5E, functioning to maintain the device in its primary folded configuration until reaching proper conditions for deployment within the GI tract. The device can be further encased within a biodegradable capsule 118.

In order to obtain further compactization in the collapsed state, the device, by some embodiments, may have has a secondary, rolled configuration (not shown), whereby the folded device is further rolled about an axis thereof, and is configured to simultaneously undergo unrolling and unfolding during transition from the collapsed state to the expanded state. It is noted that an enteric envelope can envelope the device in its secondary folded configuration (not shown), instead or in addition to the enteric envelope 122 enveloping the device in its primary folded configuration. In cases where the device includes two enteric envelopes, the first and second envelopes can be configured to have the same disintegration/dissolution properties or different disintegration/dissolution properties.

Shown in FIGS. 6A-6B is a device or an arrangement according to another embodiment of this disclosure, that also includes a deployment unit. For ease of viewing, only the general contour of the device is shown. Arrangement 300, which includes the device 100′ in its collapsed and folded configuration (similar to the folded device of FIG. 5A), is associated with a deployment unit 302, that is positioned between the folds of the device (FIG. 6A). Deployment unit 302 comprises a liquid-permeable casing 304 and at least one gas-forming material 306 contained therein, such that contact of liquid with said gas-forming material causes release of gas 308 to inflate the deployment unit (as exemplified by arrows 310), to assist in switching of device 100′ to its unfolded configuration (as seen in FIG. 6B). Typically, the rate of reaction of the gas-forming material encased in casing 304 is faster than the rate of expansion of the gel forming material in the compartments. In other words, the timing of operation of the gas-forming material is faster than that of the gel-forming material. Initial unfolding of the device is facilitated by inflation of the deployment unit, while further unfolding and expansion from the collapsed to the expanded state is achieved by expansion of the gel forming material encased in the compartments of the device.

Seen in FIGS. 6C-6D is an arrangement 400 for a device 100″ similar to the folded device of FIG. 5B, with the functional features similar to those in FIGS. 6A-6B (shifted by 100). The devices of FIGS. 6A-6B and FIGS. 6C-6D differ in their primary folded configuration.

While the examples provided herein discuss deployment of the device in the intestine, it is to be understood that the device can be administered and deployed in any other suitable bodily lumen or cavity. For example, the device can be administered to other organs, such as urine tract, vagina, rectally, intranasally, etc. Where the target organ or cavity is relatively user-accessible, the device can be administered by utilizing a dedicated applicator (not shown), in order to insert the device into the organ or cavity. The properties of the water-disintegrable sealing composition are then tailored to the specific conditions at the desired target site.

1-62. (canceled)

63. An ingestible self-expandable device, the device having a collapsed state and an expanded state, and comprising:
two or more self-expandable compartments, connected to one another by connecting zones, each compartment being formed out of two, substantially water-insoluble deformable film portions, the two film portions being bonded to each other at peripheral segments of the compartment by at least one water-disintegrable sealing composition to define sealing zones, each sealing zone being a layered structure comprised of said water-insoluble deformable films sandwiching between them at least one water-disintegrable sealing composition layer that bonds said water-insoluble deformable films to one another at the peripheral segments,
the two film portions defining between them, at areas enclosed by the peripheral segments, an enclosed space of the compartment,
each compartment having one or more liquid-permeable sections and a gel forming material within said enclosed space, the gel forming material being configured for swelling upon contact with liquid, thereby expanding the compartment to irreversibly switch the device from the collapsed state to the expanded state,
the water-disintegrable sealing composition provides mechanical stability to the sealing zones during transition of the device from the collapsed state to the expanded state, and having water-solubility configured to provide controlled disintegration of the sealing zones after the device is expanded into the expanded state, to thereby cause loss of integrity of the compartments and/or the device after its deployment.

64. The device of claim 63, wherein the connecting zones comprise said sealing zones.

65. The device of claim 63, wherein adjacent compartments are connected one to the other by bonding one or more of their respective sealing zones by a water-disintegrable sealing composition.

66. The device of claim 65, wherein said compartments are connected one to the other by stacking sealing zones of adjacent compartments one over the other, such that the connecting zones consist of the stacked sealing zones, to form a layered structure of alternating layers of said water-insoluble deformable films and the water-disintegrable sealing compositions.

67. The device of claim 65, wherein the sealing zones comprise a first water-disintegrable sealing composition, and the adjacent compartments are connected one to the other by bonding one or more of their respective sealing zones by a second water-disintegrable sealing composition, the first and second water-disintegrable sealing compositions being different one from the other or being the same.

68. The device of claim 63, wherein said water-disintegrable sealing composition comprises at least one first hydrophilic material and at least one second hydrophilic material,
the first hydrophilic material being less hydrophilic than the second hydrophilic material; and
the first hydrophilic material being less water-soluble than the second hydrophilic material.

69. The device of claim 68, wherein said first hydrophilic material being chemically and/or thermodynamically compatible with the water-insoluble deformable film, for bonding of the water-insoluble deformable films one to the other at the sealing zones.

70. The device of claim 68, wherein the amount of the first hydrophilic material in the water-disintegrable sealing composition is larger than the amount of said second hydrophilic material.

71. The device of claim 63, wherein said water-disintegrable sealing composition is located within a region of the sealing zone.

72. The device of claim 63, comprising at least one tissue-attachable element connected to at least a portion of an external surface of at least one of the compartments, such that expansion of the device from the collapsed state to the expanded state causes expansion of the compartment to drive the tissue-attachable element towards a tissue of the gastrointestinal tract, for attaching at least part of the tissue-attachable element to the tissue.

73. The device of claim 72, wherein the tissue-attachable element comprises at least one mucoadhesive material, a mucoadhesive layer, or a tissue-attachable layer.

74. The device of claim 73, wherein the mucoadhesive layer comprises at least one mucoadhesive material and at least one pharmaceutical active agent.

75. The device of claim 63, comprising at least one active agent carrying element connected to at least a portion of an external surface of at least one of the compartments, such that expansion of the device from the collapsed state to the expanded state causes expansion of the compartment to drive the active agent carrying element towards a tissue of the gastrointestinal tract and hold the active agent carrying element in contact with the tissue while the device is in the expanded state.

76. The device of claim 75, wherein the active agent carrying element is in the form of a layer comprising at least one active agent and coating at least a portion of the external surface of the compartment.

77. The device of claim 75, wherein the active agent carrying element is a solid composition or a tablet comprising at least one active agent.

78. The device of claim 63, wherein, when in the collapsed state, the device is folded in a primary folded configuration and is configured to undergo unfolding during transition from the collapsed state to the expanded state.

79. The device of claim 78, comprising at least one deployment unit, positioned between folded sections of the device when in said primary folded configuration, and configured to expand upon contact with liquid to assist to unfold of the device.

80. The device of claim 79, wherein said deployment unit comprises a liquid-permeable casing forming a closed structure and at least one gas-forming material contained therein.

81. The device of claim 79, wherein, when in the collapsed state, the device has a secondary, rolled configuration, whereby the folded device is further rolled about an axis thereof, and is configured to simultaneously undergo unrolling and unfolding during transition from the collapsed state to the expanded state.

82. An ingestible self-expandable device configured for attaching a tissue-attachable element to a tissue, the device having a collapsed state and an expanded state, and comprising:
two or more self-expandable compartments, connected to one another by connecting zones, each compartment being formed out of two, substantially water-insoluble deformable film portions, the two film portions being bonded to each other at peripheral segments of the compartment by at least one water-disintegrable sealing composition to define sealing zones, each sealing zone being a layered structure comprised of said water-insoluble deformable films sandwiching between them at least one water-disintegrable sealing composition layer that bonds said water-insoluble deformable films to one another at the peripheral segments,
the two film portions defining between them, at areas enclosed by the peripheral segments, an enclosed space of the compartment,
each compartment having one or more liquid-permeable sections and a gel forming material within said enclosed space, the gel forming material being configured for swelling upon contact with liquid, thereby expanding the compartment to irreversibly switch the device from the collapsed state to the expanded state;
the water-disintegrable sealing composition provides mechanical stability to the sealing zones during transition of the device from the collapsed state to the expanded state, and having water-solubility configured to provide controlled disintegration of the sealing zones after the device is expanded into the expanded state, to thereby cause loss of integrity of the compartments and/or the device after its deployment, and
at least one tissue-attachable element attached to at least a portion of an external surface of at least one of the compartments,
such that expansion of the device from the collapsed state to the expanded state causes expansion of the compartment to drive the tissue-attachable element towards said tissue for attaching at least part of the tissue-attachable element to the tissue.

83. An ingestible self-expandable device configured for delivery of at least one active agent to a tissue, the device having a collapsed state and an expanded state, and comprising:
two or more self-expandable compartments, connected to one another by connecting zones, each compartment being formed out of two, substantially water-insoluble deformable film portions, the two film portions being bonded to each other at peripheral segments of the compartment by at least one water-disintegrable sealing composition to define sealing zones, each sealing zone being a layered structure comprised of said water-insoluble deformable films sandwiching between them at least one water-disintegrable sealing composition layer that bonds said water-insoluble deformable films to one another at the peripheral segments,
the two film portions defining between them, at areas enclosed by the peripheral segments, an enclosed space of the compartment, each compartment having one or more liquid-permeable sections and a gel forming material within said enclosed space, the gel forming material being configured for swelling upon contact with liquid, thereby expanding the compartment to irreversibly switch the device from the collapsed state to the expanded state;
the water-disintegrable sealing composition provides mechanical stability to the sealing zones during transition of the device from the collapsed state to the expanded state, and having water-solubility configured to provide controlled disintegration of the sealing zones after the device is expanded into the expanded state, to thereby cause loss of integrity of the compartments and/or the device after its deployment, and
at least one active agent carrying element attached to at least a portion of an external surface of at least one of the compartments,
such that expansion of the device from the collapsed state to the expanded state causes expansion of the compartment to drive the active agent carrying element towards said tissue and hold the active agent carrying element against the tissue for a predefined period of time to permit delivery of the active agent from the active agent carrying element to the tissue.
